(19) United States
(12) Patent Application Publication
(10) Pub. No.: US 2026/0165335 A1
(43) Pub. Date: Jun. 18, 2026

(54) STARTER CULTURE FOR THE FERMENTATION OF A PLANT-BASED MILK ALTERNATIVE

(71) Applicant: VF Nutrition GmbH, Berlin (DE)

(72) Inventors: Anna Birke, Berlin (DE); Moritz Braunwarth, Berlin (DE); Nicolas Hartmann, Berlin (DE); Ricarda Kellermann, Melle (DE); Yifan Zhang, Berlin (DE)

(73) Assignee: VF Nutrition GmbH, Berlin (DE)

(21) Appl. No.: 19/416,934

(22) Filed: Dec. 11, 2025

(30) Foreign Application Priority Data
Dec. 13, 2024 (DE) 102024137707.0

(51) Int. Cl. A23C 11/10; C12R 1/225

(52) U.S. Cl. CPC A23C 11/106; C12N 1/205; A23V 2400/175; A23V 2400/215; A23V 2400/231; C12R 2001/225

(57) ABSTRACT

A starter culture for the fermentation of a plant-based milk alternative is provided which is particularly suitable for the manufacture of a plant-based yogurt alternative product. The starter culture comprises lactic acid bacteria isolated from leguminous plant material, in particular from pea plant material. Preferably, the starter culture comprises lactic acid bacteria of the species Lactobacillus rhamnosus and/or of the species Lactococcus lactis ssp. cremoris.

The present invention relates to a starter culture for the fermentation of a plant-based milk alternative and in particular for the manufacture of a plant-based yogurt alternative product, a method for manufacturing a fermented plant-based milk alternative product, and a fermented plant-based milk alternative product which can be manufactured using the starter culture. The invention also relates to a method for providing a starter culture with microorganisms for the fermentation of a plant-based milk alternative.

### FIELD OF APPLICATION AND PRIOR ART

The demand for the development of plant-based foods has seen an enormous increase in recent years. Non-dairy alternative products play a major role alongside meat alternatives. Soybeans are used extensively as the basis for non-dairy alternative products and, above all, for yogurt product alternatives, which currently account for the third-largest market share of plant-based foods in Europe but are controversial from an ecological and ethical point of view. Nut, coconut, rice, and oat products, for example, represent an alternative to soy protein-based products, particularly in the area of yogurt alternatives.

Patent application US 2022/0394988 A1 describes a method for manufacturing a fermented, plant-based foodstuff in which Lactococcus species are used for fermentation.

Patent application US 2022/0053788 A1 describes a method for manufacturing a plant-based fermented foodstuff that is based, for example, on a combination of soy and cashew.

Apart from soy protein-based yogurt alternatives, however, most plant-based yogurt alternatives have a nutritional profile that is characterized by a significantly higher carbohydrate content and a much lower protein content compared to yogurt based on cow's milk (Boukid, F. et al. (2021) ‘Vegan alternatives to processed cheese and yogurt launched in the European market during 2020: A nutritional challenge?’, Foods, 10(11), pp. 1-11).

Some plants that are of particular interest for the manufacture of non-dairy alternative products are representatives of the Fabaceae (legumes), for example Pisum sativum, the yellow split pea. The plant provides a nutrient-rich source of protein and can be grown in an environmentally friendly manner.

Peas are also nutritionally attractive because they have a high protein content of around 20%-35%, a low fat content of around 0.5%-4.0%, and a high starch content of around 30%-48% (Arteaga, V. G. et al. (2021) ‘Screening of twelve pea (Pisum sativum 1.) cultivars and their isolates focusing on the protein characterization, functionality, and sensory profiles’, Foods, 10(4)). They are also rich in free amino acids, complex carbohydrates, and vitamins such as vitamin B1 and B2 (Urbano, G. et al. (2003) ‘Nutritional evaluation of pea (Pisum sativum L.) Protein diets after mild hydrothermal treatment and with and without added phytase’, Journal of Agricultural and Food Chemistry, 51(8), pp. 2415-2420).

Another representative of the Fabaceae that is of particular interest in connection with the manufacture of plant-based yogurt alternatives is the broad bean (Vicia faba).

Similarly as with the manufacture of milk-based yogurt, a plant-based milk alternative based on a plant protein source (plant milk) is first provided for the manufacture of a plant-based yogurt alternative product. This plant milk is subjected to fermentation by adding a microbial starter culture, resulting in a yogurt-like texture.

Fermentation is a food technology process in which food is preserved by means of anaerobic microbiological metabolic processes. On the one hand, this occurs through the production of organic acids by the microbial culture, which leads to a drop in the pH value (acidification). On the other hand, some strains can produce bioactive metabolites with antimicrobial properties that extend the shelf life of products.

Until now, it has been common practice to use commercial starter cultures for the fermentation of plant-based milk alternatives, which are also used in classic lactic acid fermentation in the dairy industry. These starter cultures can be “veganized” by “acclimatizing” the starter cultures to plant proteins as part of a switch to plant-based dairy alternatives.

Nevertheless, such veganized starter cultures, particularly plant-based milk alternatives based on pea or broad bean proteins, cause problems in the fermentation of plant-based milk alternatives. These problems have a negative impact on manufacturing itself and on the sensory profile of the end product. In particular, long fermentation times of around 16 hours are required during manufacturing, whereas the fermentation of yogurt made from cow's milk usually only takes eight hours. Furthermore, the resulting product does not have the same positive properties as a yogurt product based on cow's milk, meaning that consumer acceptance is often unsatisfactory. Above all, there is an insufficient reduction of off-flavors and aromas (collectively referred to as “off-flavors”). These off-flavors have a negative impact on the sensory profile of the end product. For example, the hexanal content is too high. Furthermore, there is insufficient production of desirable aromas and flavors, such as acetaldehyde, diacetyl, and acetoin in particular. This generally necessitates the subsequent addition of the desired aromas and flavors to the product in the form of additives. In addition, an unsatisfactory degradation of antinutritional secondary plant metabolites which inhibit fermentation is observed.

Overall, the conventional processing of pea and/or broad bean-based protein sources into plant-based yogurt alternatives generally leaves unwanted aromas and flavors in the end product, which have a negative impact on the sensory profile. On the other hand, desirable aroma- and flavor-active substances that make up the typical yogurt profile are either not produced at all or in excessively low concentrations during fermentation. Furthermore, the conventional fermentation of plant-based milk alternatives based on pea or broad bean proteins results in insufficient acidification of the product. This is presumably due to insufficient acid production in the crop combined with the increased buffering capacity of pea and broad bean proteins.

A great need therefore exists, particularly when using pea or broad bean-based ingredients, to increase consumer acceptance of the products made from them by improving their appearance, texture, aroma, and taste profile.

### Object and Solution

Against this background, it is the object of the invention to provide an improved possibility for the fermentation of a plant-based milk alternative, in particular for the manufacture of plant-based yogurt alternatives. Above all, it is the object of the invention to provide especially suitable starter cultures for the fermentation of plant-based milk alternatives.

This object is achieved by a starter culture for the fermentation of a plant-based milk alternative which is characterized in that the starter culture comprises lactic acid bacteria isolated from leguminous plant material. Preferably, the lactic acid bacteria can be isolated from pea plant material, in particular from pea seeds and/or pea pods. In other embodiments, the lactic acid bacteria may, for example, be isolated from broad bean plant material. This starter culture with lactic acid bacteria isolated from such plant material is especially suitable for the manufacture of a plant-based yogurt alternative product.

With this approach, the inventors were able to generate a starter culture that is especially suitable for the fermentation of plant-based milk alternatives, especially milk alternatives based on pea and/or broad bean protein.

In contrast to the approach that has usually been taken up to now, in which starter cultures from the conventional dairy industry are adapted to plant proteins, the present invention takes the approach of isolating the lactic acid bacteria for the starter culture directly from the plant material. Since the plant material is the natural habitat of these microorganisms, they are specially adapted to macro- and micronutrients from this plant material and can therefore metabolize them in an especially advantageous manner. This means that the starter culture according to the invention can reduce off-flavor substances from the plant-based protein. At the same time, fermentation with the starter culture according to the invention ensures a creamy texture in the fermented product. At the same time, fermentation with the starter culture according to the invention produces substances relevant for the sensory profile, enabling the addition of natural flavors, for example, to be dispensed with.

In an especially preferred manner, the lactic acid bacteria are representatives of the species Lactobacillus rhamnosus and/or the species Lactococcus lactis ssp. cremoris.

By using different bacterial strains isolated from the plant material, the natural metabolic diversity of the microbial community from the plant material can be used to advantage in the fermentation of a plant-based milk alternative. The combination of Lactobacillus rhamnosus and Lactococcus lactis ssp. cremoris in the starter culture according to the invention is therefore especially preferred.

Preferably, the lactic acid bacteria of the species Lactobacillus rhamnosus are representatives of strain P11 (depository: DSMZ, deposit reference number: DSM 35210).

The lactic acid bacteria of the species Lactococcus lactis ssp. cremoris are preferably representatives of strain P1 (depository: DSMZ, deposit reference number: DSM 35209).

As part of the work on which this invention is based, various lactic acid bacteria were isolated from legume seeds, characterized, and examined with regard to their suitability as a starter culture, in particular for the manufacture of a yogurt alternative product based on legume protein.

The phenotypic characteristics of the bacterial strains were examined in particular with regard to growth on and utilization of pea and broad bean protein. A thickening of the matrix through fermentation and an acidification of the matrix were also considered. In addition, the degradation of off-flavor substances, particularly hexanal, and the manufacture of so-called “yogurt flavors” were included.

During this work, a Lactobacillus rhamnosus strain (strain P11, depository: DSMZ, deposit reference number: DSM 35210) was identified which had been isolated from a fresh sugar snap pea pod. This strain is characterized in that it grows excellently on medium containing mainly pea or broad bean protein isolate. In a thickening test, this strain led to a thickening of the matrix under almost all tested conditions; this was not due to the production of acid, but to the production of so-called protein crosslinkers. One of these crosslinking agents is acetaldehyde, which is both a yogurt flavoring and the precursor molecule of the two yogurt flavorings diacetyl and acetoin.

The inventors were also able to show that strain P11 reduces hexanal during fermentation, even in a co-culture with other acidifying bacterial species. Hexanal and other aldehydes as well are also responsible for off-flavors, particularly for a beany taste, in conventional plant-based milk alternative products. By reducing the hexanal content as a result of fermentation with the starter culture according to the invention, the resulting taste of the fermented product can thus be significantly improved compared to conventionally manufactured milk alternative products.

Furthermore, strain P11 was shown to degrade phytic acid in an agar-based assay. Phytic acid is a bioactive substance that is found in legumes, among other things. Due to its complexing properties, it can inhibit the absorption of minerals ingested with food, making phytic acid potentially disadvantageous nutritionally. The degradation of this bioactive plant substance by strain P11 is therefore very beneficial for the resulting milk alternative product.

In the course of this work, trial fermentations were carried out with all isolated bacterial strains and analyzed by sensory analysis. In addition to the P11 strain of Lactobacillus rhamnosus, another strain (P1) proved to be especially beneficial sensorily. The bacterial species of this strain P1 (depository: DSMZ, deposit reference number: DSM 35209) was identified as Lactococcus lactis ssp. cremoris.

Especially good results can thus be achieved during fermentation through a combination of strains P1 and P11 in a starter culture.

In another, especially preferred embodiment of the starter culture according to the invention, the starter culture additionally comprises lactic acid bacteria of the species Streptococcus thermophilus. In this context, acidifying strains and preferably strongly acidifying strains of Streptococcus thermophilus are especially preferred.

The inventors' investigations had shown that the analyzed strains of Lactobacillus rhamnosus and Lactococcus lactis ssp. cremoris in combination with an acidifying strain of Streptococcus thermophilus produce a milkiness in the fermented milk alternative product that is clearly superior to a fermentation product with conventional starter cultures.

Preferably, the plant-based milk alternative is manufactured on the basis of pea protein and/or broad bean protein.

In especially preferred embodiments, the starter culture according to the invention comprises Lactobacillus rhamnosus P11 (function: off-flavor reduction and degradation of antinutritional ingredients, thickening by crosslinker production), Lactococcus lactis ssp. cremoris P1 (function: manufacturing of milk and yogurt flavors) and Streptococcus thermophilus (acidification). This starter culture is very especially suitable for the fermentation of plant-based milk alternatives for the manufacture of yogurt alternative products based on pea and/or broad bean proteins.

The acidifying strain of Streptococcus thermophilus can be optionally isolated from milk or another food. It may even be especially advantageous to use a traditional yogurt strain from a conventional starter culture for dairy products as the acidifying strain of Streptococcus thermophilus in order to ensure particularly good acidification in this co-culture during fermentation with the starter culture according to the invention.

The invention also includes a method for manufacturing a fermented plant-based milk alternative product. This method is characterized in that the plant-based milk alternative product is fermented using a starter culture as described above. Preferably, the starter culture comprises lactic acid bacteria of the species Lactobacillus rhamnosus and/or of the species Lactococcus lactis ssp. cremoris and preferably also lactic acid bacteria of the species Streptococcus thermophilus. The lactic acid bacteria of the species Lactobacillus rhamnosus are preferably representatives of strain P11. The lactic acid bacteria of the species Lactococcus lactis ssp. cremoris are preferably representatives of strain P1, with both strain P11 and strain P1 being isolated from leguminous plant material.

In especially preferred embodiments of the manufacturing method, the plant-based milk alternative product to be manufactured is a yogurt alternative product.

In advantageous embodiments of the manufacturing method, the method comprises the following method steps:
a. provision of a plant-based milk alternative;
b. adding the starter culture to the plant-based milk alternative;
c. fermenting the plant-based milk alternative;
d. optionally, further processing the plant-based milk alternative product;
e. filling and packaging the plant-based milk alternative product.

Preferably, the plant-based milk alternative is a milk alternative based on pea protein and/or broad bean protein.

Fermentation preferably takes place at a temperature between 35 and 45° C. A temperature of 40° C. is especially preferred.

The fermentation is preferably carried out for a period of 5 to 20 hours, preferably for a period of 8 to 16 hours.

In especially preferred embodiments, fermentation or incubation takes place at 40° C. for 8 to 16 hours.

For example, a yogurt base with a pea and/or broad bean protein content of 5% can be manufactured to provide the plant-based milk alternative. This yogurt base or plant-based milk alternative is pasteurized. The starter culture according to the invention is then added. Fermentation takes place, for example, at 40° C. for 8 to 16 hours without stirring. It can be optionally stirred in order to achieve a consistency typical of yogurt products. The product is acidified as a result of the fermentation. If further acidification is required—in order to comply with legislation governing the food industry, for example—acid can be optionally added. For example, it may make sense to lower the pH value to pH 4.5 or lower if this acidification was not achieved by fermentation. The end product can then be bottled, optionally after further processing (e.g., addition of fruit components or the like). The end product is expediently cooled in order to minimize further activity of the yogurt cultures.

The invention also includes a fermented plant-based milk alternative product which is characterized in that the fermented plant-based milk alternative product is fermented using a starter culture as described above. Preferably, the starter culture comprises lactic acid bacteria of the species Lactobacillus rhamnosus and/or of the species Lactococcus lactis ssp. cremoris and preferably also lactic acid bacteria of the species Streptococcus thermophilus. The lactic acid bacteria of the species Lactobacillus rhamnosus are preferably representatives of strain P11. The lactic acid bacteria of the species Lactococcus lactis ssp. cremoris are preferably representatives of strain P1.

In especially preferred embodiments of the plant-based milk alternative product, the plant-based milk alternative product is a yogurt alternative product.

Preferably, the milk alternative product and, in particular, the yogurt alternative product, is based on a milk alternative based on pea protein and/or broad bean protein.

By virtue of using the starter culture according to the invention in the manufacture of the plant-based milk alternative product, the milk alternative product has various highly advantageous features. In particular, the fermented plant-based milk alternative product is characterized by a thickening and/or an acidification and/or a reduced content of off-flavor substances and/or an increased content of milk and/or yogurt flavorings and/or a reduced content of antinutritional ingredients.

The off-flavor substances that can be reduced by fermentation with the starter culture according to the invention compared to fermentation with conventional starter cultures are particularly hexanal.

The milk and/or yogurt flavorings which may be present in greater quantities in the resulting product compared to conventional starter cultures are particularly the aroma compounds acetaldehyde and/or diacetyl and/or acetoin.

The antinutritional ingredients which may be present in lesser quantities in the resulting product compared to conventional starter cultures are, in particular, fermentation-inhibiting secondary plant metabolites such as phytic acid.

In especially preferred embodiments of the fermented plant-based milk alternative product, the milk alternative product is free of added flavorings. By virtue of the special advantages described, which are achieved by using the starter culture according to the invention during fermentation, it is possible to preferably completely dispense with the addition of flavorings, since the resulting product has a very pleasant sensory profile for the consumer even without such additives.

Preferably, a total of more than¹⁰⁸ microorganisms are present in the fermented plant-based milk alternative product, with Lactobacillus rhamnosus and Streptococcus thermophilus preferably being present in a ratio of approximately 1:1.

In experiments, the inventors observed that fermentation with the starter culture according to the invention resulted in greater acidification of the product than with a conventional starter culture. This represents a further advantage of the starter culture according to the invention, since acidification is an essential function of fermentation.

Finally, the invention includes a method for providing a starter culture with microorganisms, in particular with lactic acid bacteria and/or yeasts, preferably with lactic acid bacteria, for the fermentation of a plant-based milk alternative based on a plant-based protein source from a defined plant-based material. This method is characterized in that the defined plant material is used to isolate the microorganisms.

Especially advantageous embodiments of the method comprise the following method steps:
a. provision of a sample of the defined plant material;
b. enrichment and isolation of microorganisms, in particular gram-positive bacteria, from the sample of plant material;
c. optionally, genotaxonomic identification of the microorganisms;
d. phenotypic characterization of the microorganisms;
e. performing trial fermentations of the plant-based milk alternative with the isolated microorganisms;
f. selecting and compiling suitable microorganisms based on the results of the trial fermentations for the provision of a starter culture.

In preferred embodiments of the method, the phenotypic characterization of the microorganisms comprises at least one of the following aspects:
a. thickening of a matrix;
b. acidification of a matrix;
c. degradation of off-flavor substances, particularly hexanal;
d. production of milk and/or yogurt flavorings, in particular acetaldehyde and/or diacetyl and/or acetoin;
e. usability of different sugar sources;
f. influence of different sugar sources on metabolite production.

In especially advantageous embodiments of the method, the defined plant material is plant material of a representative of the Fabaceae (leguminous plants). Especially preferred representatives of the Fabaceae are Pisum sativum and/or Vicia faba. Suitable plant material for isolation and/or characterization are, for example, the pea pods or beans of these species.

In the following, exemplary embodiments of the invention will be explained in greater detail with reference to the drawings. The individual features can be realized individually or in combination with each other.

### BRIEF DESCRIPTION OF THE DRAWINGS

### Description of the Drawings

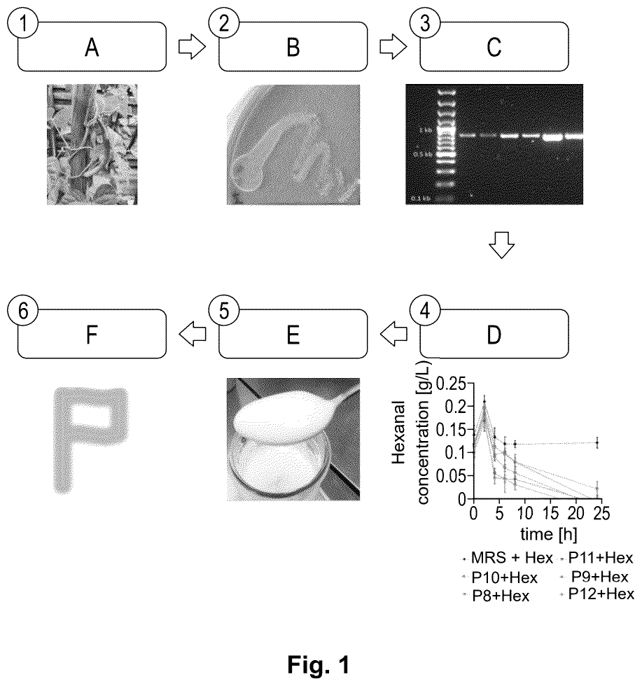

FIG. 1 Overview of the steps involved in isolating the microorganisms for the starter culture;

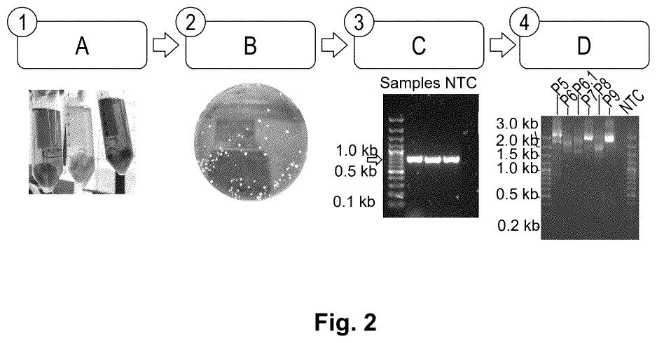

FIG. 2 Flowchart of the work steps with exemplary images for the isolation and identification of lactic acid bacteria from pea and bean samples. Kb: Kilobases; NTC: Negative PCR control (non-template control).

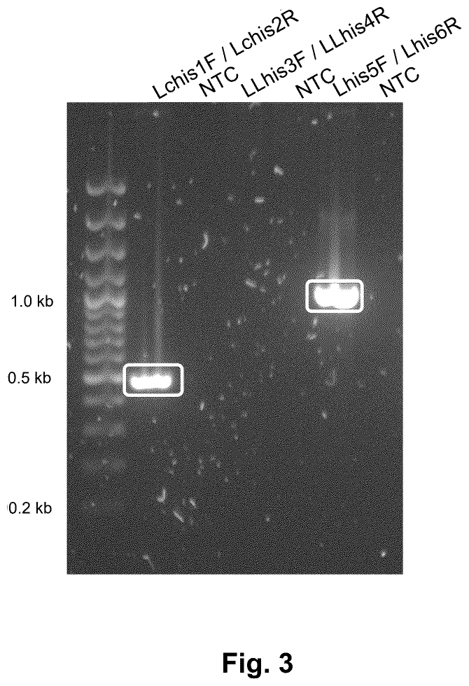

FIG. 3 Agarose gel (1.0%) with bands amplified by PCR with specific primer pairs;

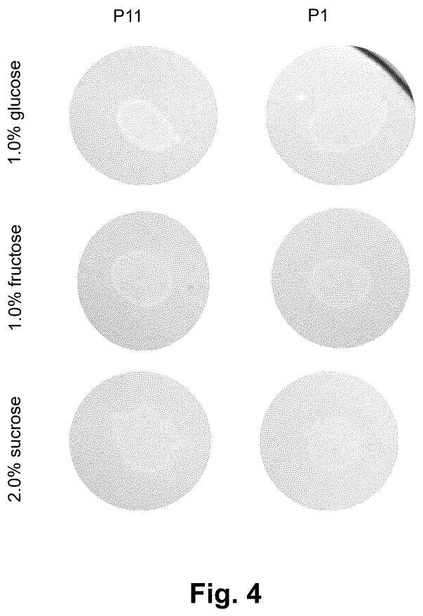

FIG. 4 Growth of strains P11 and P1 on pea protein-containing medium with different sugar sources;

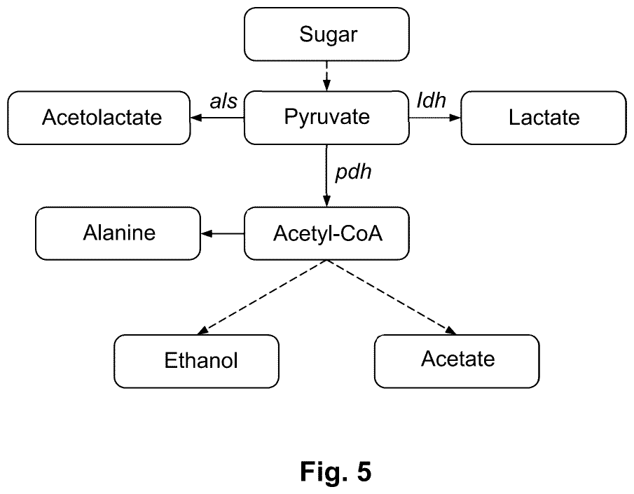

FIG. 5 Schematic of central metabolic pathways in which pyruvate serves as a precursor molecule. als: Acetolactate synthase; ldh: Lactate dehydrogenase; pdh: Pyruvate dehydrogenase;

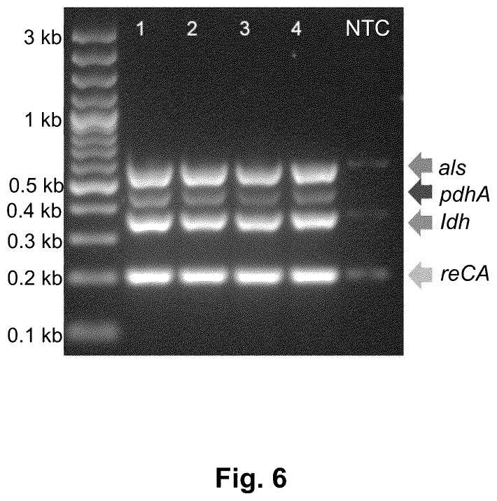

FIG. 6 Agarose gel (2.0%) for RT-PCR with gDNA as the template. 1: P11-gDNA as the template; 2: P12-gDNA as the template; 3 and 4: L. rhamnosus gDNA from strains isolated from commercial yogurt samples; NTC: Negative PCR control (non-template control);

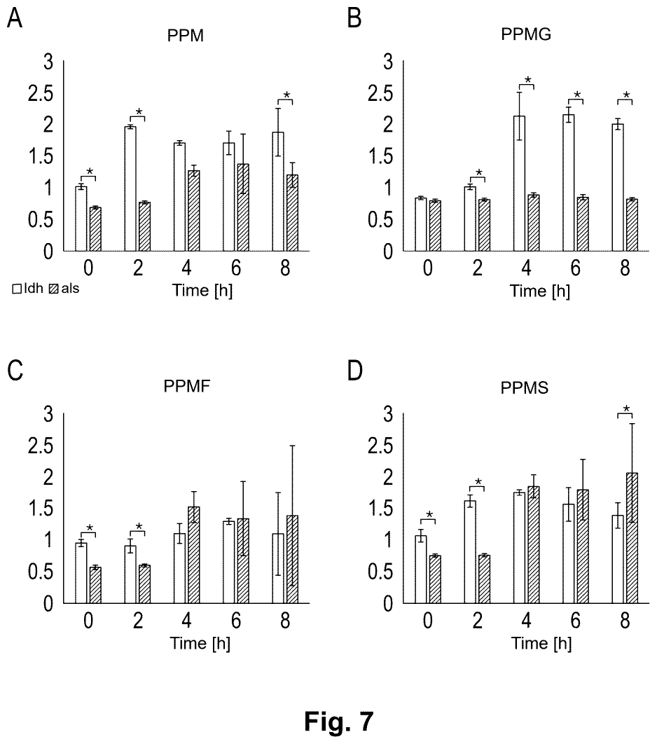

FIG. 7 Relative transcript abundance of ldh and als in three independent P11 liquid cultures at selected timepoints. The error bars show the standard deviation (*=p value below 0.05). A) PPM: Pea medium; B) PPMG: Pea medium, 1.0% glucose; C) PPMF: Pea medium, 1.0% fructose; D) PPMS: Pea medium, 2.0% sucrose;

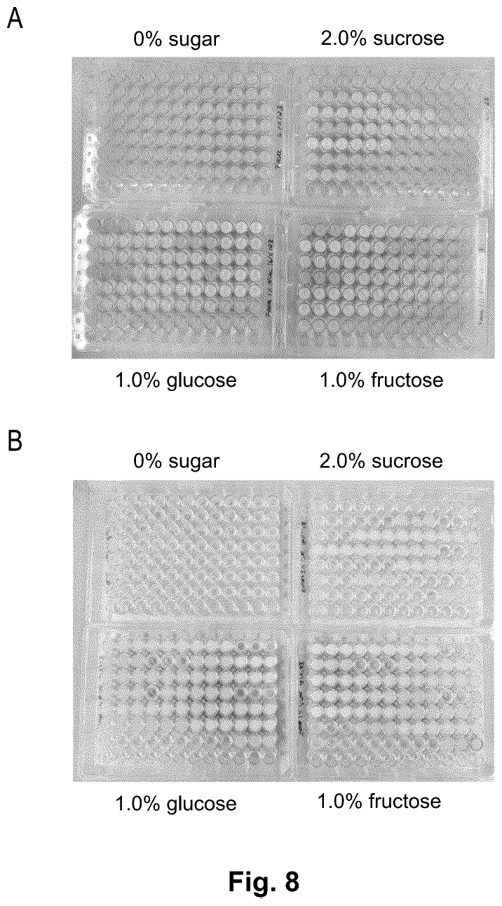

FIG. 8 Representative 96-well plates with broad bean medium before (A) and after (B) inversion;

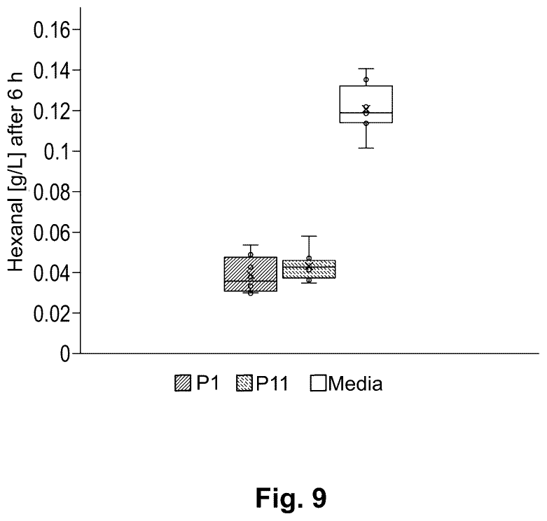

FIG. 9 Hexanal concentrations [g/L] in the fermented samples of P1 and P11 compared to the unfermented control (medium) after 6 hours of incubation at 40° C.; and

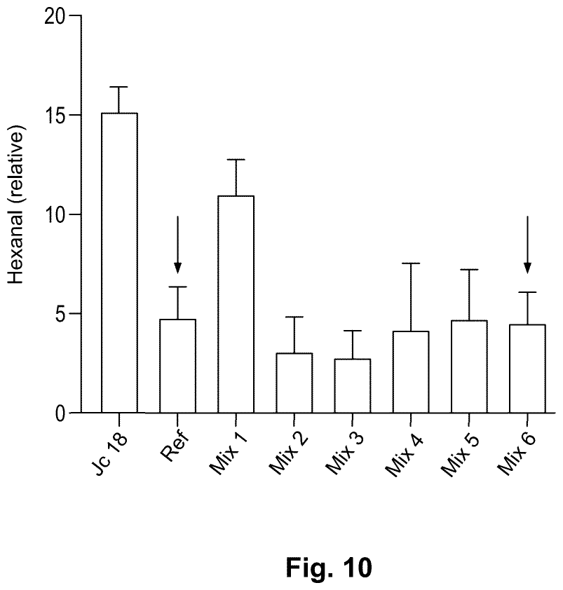

FIG. 10 Relative hexanal contents in fermented samples. JC 18: Streptococcus thermophilus, isolated from pea material; Ref: Sample fermented with the commercial starter culture; Mix 1-Mix 6: Fermentation with different strains isolated from peas in combination with a weakly acidifying St. thermophilus strain; Mix 1 contains strain P1 and Mix 6 contains strain P11.

### DESCRIPTION OF PREFERRED EXEMPLARY EMBODIMENTS

FIG. 1 shows an overview of the work steps for the isolation and characterization of lactic acid bacteria from plant material with exemplary illustrations. This method was used to isolate and characterize lactic acid bacteria from pea plants and broad bean plants and investigate their suitability for a starter culture that can be used in the manufacture of a pea or bean protein-based yogurt alternative. The various strains examined were labeled with the letter P and a consecutive number.
Step A—Collection of plant material
Step B—Enrichment and isolation of lactic acid bacteria
Step C—Genotaxonomic identification
Step D—Phenotypic characterization
Step E—Trial fermentations
Step F—Compilation of the starter culture
   Isolation and Identification of Lactic Acid Bacteria from Pea and Bean Material

For the isolation and identification of lactic acid bacteria from pea and bean material, peas and beans were first collected from seven different sources, some directly from pea fields in Bavaria, North Rhine-Westphalia, and Brandenburg. 13 strains belonging to five different lactic acid bacteria species were isolated from the sources (peas and beans).

FIG. 2 illustrates the steps involved in isolating and identifying the lactic acid bacteria.
Step A—Enrichment
Step B—Selection
Step C—Identification of the species
Step D—Subspecies/strain differentiation

Enrichment (step A): The microorganisms were enriched in liquid medium in which the growth of lactic acid bacteria is promoted. To enrich the microorganisms on the pea and bean samples, the seeds were incubated in liquid medium for up to 4 weeks at 15-18° C. under anaerobic conditions. In order to enrich both epi- and endophytic bacteria, some liquid cultures were prepared with crushed seeds. Different media were used for the enrichment, in particular M17 medium (left in step A of FIG. 2), tryptone soy yeast extract medium (TSY; center in step A of FIG. 2) and De Man-Rogosa-Sharpe medium (MRS; right in step A of FIG. 2).

Selection (step B): Once the liquid medium was visibly turbid and the optical density was measurable, small volumes were spread onto various agar plates (MRS, M17, TSY, SM). The plates were previously mixed with nalidixic acid, an antibiotic that is effective against both gram-negative and gram-positive bacteria. At low doses, however, nalidixic acid mainly inhibits the growth of gram-negative bacteria. The exemplary plate in step B of FIG. 2 shows individual colonies on MRS agar. When plated on medium without nalidixic acid, mainly enterobacteria and representatives of the genera Shigella and Clostridium grew on the plates.

Identification of the species (step C): Genotaxonomic identification of the species was performed by 16S rRNA amplification (PCR product with 0.8 kilobases). The resulting PCR products were subsequently sequenced with the same primers (see Aforijiku, S., Fakorede, C. O. and Adediran, A. B. (2021) ‘Strain-level Identification of Beneficial Lactobacilli of Dairy Origin using 16S rRNA Sequencing: A Biotechnology Approach’, Microbiology Research Journal International, 31(2), pp. 13-21). Through a database comparison with previously sequenced and identified species, the isolated bacteria were matched with a species/strain. The agarose gel in step C of FIG. 2 shows the PCR products (approx. 0.8 kilobases) that were amplified with the 16S primers.

Strain/subspecies differentiation (step D): Subspecies of the same species were determined with specific primers. Multiple strains of the same species were differentiated using random amplification of polymeric DNA (RAPD)-PCR. RAPC-PCR was performed with the universal primer M13, which binds to different sites in the genome and, depending on the genome sequence, leads to distinct band patterns, so-called fingerprints (see Rossetti, L. and Giraffa, G. (2005) ‘Rapid identification of dairy lactic acid bacteria by M13-generated, RAPD-PCR fingerprint databases’, Journal of Microbiological Methods, 63(2), pp. 135-144).

One of the bacterial species identified was Lactococcus lactis, of which three subspecies are known: Lactococcus lactis, Lactococcus lactis biovar. diacetylactis, and Lactococcus lactis ssp. cremoris. These three subspecies can be distinguished from each other with specific primer pairs that bind in the histidine biosynthesis operon (Beimfohr, C., Ludwig, W. and Schleifer, K. H. (1997) ‘Rapid genotypic differentiation of Lactococcus lactis subspecies and biovar’, Systematic and Applied Microbiology, 20(2), pp. 216-221). FIG. 3 shows the result of this PCR with specific primers. Based on the visible bands of approx. 0.5 kb and 1.0 kb, the bacterium was identified as Lactococcus lactis ssp. cremoris.

Utilization of Industrially Relevant Sugar Sources and their Influence on Metabolite Production

To investigate the preference for sugar sources, either glucose (1.0%), fructose (1.0%), or sucrose (2.0%) was added to the culture medium. For this growth comparison, approx. 8×10⁵ cells were dropped onto the agar plates and incubated anaerobically at the industrial fermentation temperature of 40° C. for two weeks. FIG. 4 shows the growth of P11 and P1 on pea protein-containing medium with these different sugar sources after a two-week incubation phase at 40° C.

Although neither the tested sugar sources nor the fermentation temperature corresponded to the natural original habitat of these strains, good growth was observed in both strains.

FIG. 5 shows a diagram with central metabolic pathways in which pyruvate serves as a precursor molecule (als: acetolactate synthase; ldh: lactate dehydrogenase; pdh: pyruvate dehydrogenase). The direct substance conversions are shown with uninterrupted arrows, while indirect substance conversions are shown with interrupted arrows. Lactic acid production (lactate production) and aroma formation (formation of acetolactate) are competing metabolic pathways.

Semi-quantitative gene expression analyses were performed in order to understand which metabolic pathways are active under which conditions in relation to the strains studied.

The genes selected encode L-lactate dehydrogenase (ldh), acetolactate synthase (als), and a pyruvate dehydrogenase subunit (pdh). The diagram in FIG. 5 shows how the enzymes compete with each other, since pyruvate is the precursor molecule for all reactions. The conversion of pyruvate to lactic acid by L-lactate dehydrogenase is the most important means of acidification in yogurt manufacturing. Acetolactate, on the other hand, is an intermediate metabolic product that can be further converted to diacetyl and acetoin, which are key flavor components of yogurt. Since L. rhamnosus is a heterofermentative bacterium, the formation of acetate and ethanol cannot be completely ruled out if sufficient pyruvate is present. Therefore, pdh was included as a key gene in the analysis.

An overview of the selected genes, the function of the respective gene product, and the size of the resulting PCR products are shown in Table 1 below.

TABLE 1
Overview of selected genes of central metabolic pathways,
with the function of the respective gene product and
the size of the PCR product in an RT-PCR.

| | | Size of the |
|---|---|---|
| Gene | Function of the gene product | PCR product |
| recA | Recombinase A | 210 bases |
| Ldh | Lactate dehydrogenase | 381 bases |
| pdhA | Pyruvate dehydrogenase E1 | 478 bases |
| | component alpha subunit | |
| Als | 2-acetolactate synthase | 604 bases |

Primer pairs that specifically amplify DNA segments within ldh, als, and pdhA were first tested with genomic DNA (gDNA) as the PCR template. FIG. 6 shows the resulting PCR products. Four different gDNA samples were used as templates, namely from P11, another strain P12, and two L. rhamnosus strains isolated from plant-based yogurt alternatives. As can be seen in the agarose gel in FIG. 6, four clear bands of the expected sizes (see Table 1) can be seen per lane. The light bands in the negative control (NTC) lane are probably due to overflowing of sample 4 during loading of the gel.

In the next step, P11 was selected and cultivated in the following liquid media: Pea protein medium without added carbohydrate source (PPM), as well as PPM with either 1.0% glucose (PPMG), 1.0% fructose, (PPMF) or 2.0% sucrose (PPMS). The cultures were each prepared in biological triplicates. First, the relative transcript levels of the key genes were compared in 24-hour-old liquid cultures. For this purpose, the RNA of the liquid cultures was isolated after 24 hours, then transcribed into cDNA using a reverse transcriptase in order to amplify the desired products in a PCR. The strength or brightness of the band on an agarose gel indicated how much of the initial template was present in the reaction. This allows inferences to be made about the relative occurrence of the respective RNA transcript.

The brightness of the bands was determined using an image processing program (imageJ). The values of the ldh and als bands were then normalized to the brightness of the corresponding recA band. RecA is a housekeeping gene whose expression remains constant and is therefore usually used for normalization in semi-quantitative gene expression analyses.

FIG. 7 shows the results of the relative transcript abundance of ldh and als in three independent P11 liquid cultures at selected timepoints. The RNA transcripts at P11 were analyzed when incubated for 24 hours in different media (pea protein medium (PPM), pea protein medium with 1.0% glucose (PPMG), pea protein medium with 1.0% fructose (PPMF), pea protein medium with 2.0% sucrose (PPMS)). Statistically significant differences were determined using the Kruskal-Wallis test (tool used: https://www.statskingdom.com/kruskal-wallis-calculator.html). An overview of the p-values determined can be found in Table 2.

TABLE 2
Overview of the p-values determined in Kruskal-Wallis (KW)
tests for the values shown in FIG. 7. ns: not significant.

| | Time | KW test |
|---|---|---|
| Medium | [h] | p-value |
| PPM | 0 | 0.00189 |
| | 2 | 0 |
| | 4 | ns |
| | 6 | ns |
| | 8 | 2.47E−06 |
| PPMG | 0 | ns |
| | 2 | 1.97E−07 |
| | 4 | 4.7E−06 |
| | 6 | 1.19E−11 |
| | 8 | 8.40E−12 |
| PPMF | 0 | 0.0000174 |
| | 2 | 0.001351 |
| | 4 | ns |
| | 6 | ns |
| | 8 | ns |
| PPMS | 0 | 0.00189 |
| | 2 | 0 |
| | 4 | ns |
| | 6 | ns |
| | 8 | 2.5E−06 |

While the relative transcript abundance of ldh in PPM and PPMG was in part significantly greater than that of als, a clear influence of glucose in PPMG on the expression of both genes could be seen. It is possible that ldh expression is stimulated and possibly suppressed by catabolite repression. Directing the carbon flow towards lactate formation in PPMG could lead to acid formation during fermentation at the expense of aroma formation.

In contrast, the sucrose contained in PPMS had a positive effect on als and ldh expression, with the limiting factor for acetolactate and lactate formation being the intracellular availability of pyruvate. This means that even if sucrose stimulates the gene expression of ldh and als, this does not mean that more carbon can be converted overall. Sucrose is a disaccharide that has to be transported into the cell and hydrolyzed. These steps crucially determine the intracellular availability of pyruvate.

Based on these data, the sugar source in the yogurt alternative product recipe was adjusted by adding a mixture of glucose and sucrose.

### Thickening of the Matrix Through Fermentation

Based on the method described in the article by Genet et al. (Genet, B. M. L. et al. (2023) ‘Selection of proteolytic LAB starter cultures for acidification of soy based dairy alternatives’, LWT, 184, p. 115082), the acid production of the lactic acid bacteria and the associated thickening of the matrix were investigated. Since thickening is an important task of the yogurt starter culture—both through the production of acid, e.g., lactic acid, and the associated precipitation of the proteins contained in the raw material, as well as through the production of exopolysaccharides—the “tiling” method presented in the article was adapted and applied in order to test the matrix thickening capability of the strains isolated from pea material. The adapted method was used to test liquid pea (PPM) and broad bean (FPM) medium in 96-well plates, which either contained no other sugar (0% sugar) or, analogously to the agar plates, contained glucose (PPMG, FPMG), fructose (PPMF, FPMF), or sucrose (PPMS, FPMS). After incubation for 14-16 hours, the plates were inverted.

FIG. 8 shows a representative 96-well plate with broad bean medium (FPM) before (A) and after (B) inversion. Only FPM that had thickened during incubation was still visible in the wells after inversion (negative control: FPM with water). Wells in which the medium was not thickened were empty after inverting, as was the case for all strains when no added sugar was present in the medium. Some of the wells were thickened with added sugar, which could also be seen from the lighter color.

Strain P11 always thickened the matrix when either glucose or fructose was present. P1, on the other hand, the pea protein matrix thickened only with added fructose, and the broad bean protein matrix thickened with all tested sugar sources. P11 produced the firmest gel in PPMG. However, the thickening cannot be attributed primarily to acid formation, as the pH was lowered only minimally. This observation was later confirmed on a larger scale in the trial fermentations.

### Reduction of Off-Flavors (Hexanal)

Hexanal is an aldehyde that contributes to the grassy, green off-flavor in plant-based foods. Furthermore, it can be considered as a representative for aldehydes formed by lipid oxidation in pea and broad bean protein isolates, whose off-flavors are perceived as objectionable in the overall sensory profile (for example, see Roland, W. S. U. et al. (2017) ‘Flavor aspects of pulse ingredients’, Cereal Chemistry, 94(1), pp. 58-65).

The method used to measure the reduction in hexanal content was adapted based on the method described by Kozaeva et al. for the quantification of hexanal in liquid medium (see Kozaeva, E. et al. (2022) ‘High-throughput colorimetric assays optimized for detection of ketones and aldehydes produced by microbial cell factories’, Microbial Biotechnology, 15(9), pp. 2426-2438). For this purpose, 0.2 g/L hexanal was added to the MRS medium and inoculated, and the absorbance was measured at a wavelength of 630 nm. The hexanal concentrations in the fermented samples were determined using a calibration curve. Since 630 nm is not a wavelength at which it can be ruled out that residues of the culture after centrifugation or other substances in the medium are not also being measured, identical cultures without hexanal were also analyzed at 630 nm.

Although absorbance values>0 were measured in the control cultures without the addition of hexanal, the values were constant over the measurement period (not shown), unlike for the calculated hexanal values of the fermented samples. For these samples, a significant decrease was observed after four hours compared to the medium control (MRS plus 0.15 g/L hexanal). The difference in hexanal concentrations between the fermented samples and the medium control increased after four hours and reached a maximum at 24 hours.

After 6 hours, clear differences between the strains were recognizable. As can be seen in FIG. 9, after this time there was also a clear difference between the samples fermented with P1 and P11 compared to the unfermented medium control. It was therefore shown that fermentation with strains P1 and P11 significantly reduced the hexanal concentration in the medium.

### Trial Fermentations

Various trial fermentations were carried out (JC 18-Streptococcus thermophilus, isolated from pea material, Ref—fermentation with commercial starter culture, Mix 1—fermentation with strain P1, Mix 2—fermentation with another strain from pea material, Mix 3—fermentation with another strain from pea material, Mix 4—fermentation with another strain from pea material, Mix 5—fermentation with another strain from pea material, Mix 6—fermentation with strain P11), and the relative hexanal content was determined in each case.

FIG. 10 shows the results of the hexanal assay. The first round of trial fermentations showed that strain P11 in co-culture with a weakly acidifying Streptococcus thermophilus strain (Mix 6) was able to reduce the hexanal content as well as the commercial reference did.

The culture in Mix 1 contained P1 and the weakly acidifying Streptococcus thermophilus strain. As can be seen in FIG. 10, the hexanal content in this sample was higher than in the reference sample.

In addition to the chemical analyses, the fermented samples were also tasted (see Table 3). In order to better evaluate the influence of the starter cultures, no masking or yogurt flavors were added to the basic recipe of the “starting pea milk”, which is why the evaluation of the reference sample was rather negative.

TABLE 3
Overview of aroma and taste impressions gained during
the tasting of the samples. In this context, “cardboardy”
refers to a “cardboard-like” taste.

| Sample | Aroma impressions | Taste impressions |
|---|---|---|
| Ref | Beany, grassy | Bitter, cardboardy, metallic |
| Mix 1 | Slightly beany | Better than Ref., milky |
| Mix 2 | Slightly beany | Relatively neutral, bitter, cardboardy |
| Mix 3 | Beany, grassy | Metallic, astringent, bitter |
| Mix 4 | beany | Umami, salty |
| Mix 5 | beany, grassy | Too salty, cardboardy |
| Mix 6 | Neutral, slightly sour | Milky, pleasantly tart, slightly |
| | | cardboardy |

Overall, Mix 1 (strain P1) and Mix 6 (strain P11) had the best sensory profiles in the tasting. These results show that the fermentation with strain P1 and the fermentation with strain P11 achieved the best sensory result in comparison with other strains isolated from pea material and in comparison with a conventional starter culture (Ref). In terms of hexanal content, strain P1 was slightly worse than the conventional starter culture. However, strain P11 achieved a reduction in hexanal content comparable to the reference. A combination of strains P1 and P11 together with an acidifying Streptococcus thermophilus strain can therefore achieve good hexanal reduction with a superior sensory profile.

1. A starter culture for the fermentation of a plant-based milk alternative, in particular for the manufacture of a plant-based yogurt alternative product, characterized in that the starter culture comprises lactic acid bacteria isolated from leguminous plant material, in particular from pea plant material.

2. The starter culture according to claim 1, characterized in that the lactic acid bacteria are representatives of the species Lactobacillus rhamnosus and/or of the species Lactococcus lactis ssp. cremoris.

3. The starter culture according to claim 1, characterized in that the starter culture comprises lactic acid bacteria of the species Lactobacillus rhamnosus which are representatives of strain P11 (depository: DSMZ, file number of the deposit: DSM 35210).

4. The starter culture according to claim 1, characterized in that the starter culture comprises lactic acid bacteria of the species Lactococcus lactis ssp. cremoris, which are representatives of strain P1 (depository: DSMZ, file number of the deposit: DSM 35209).

5. The starter culture according to claim 1, characterized in that the starter culture additionally comprises lactic acid bacteria of the species Streptococcus thermophilus.

6. The starter culture according to claim 1, characterized in that the plant-based milk alternative is manufactured on the basis of pea protein and/or broad bean protein.

7. A method for the preparation of a fermented plant-based milk alternative product, characterized in that the plant-based milk alternative product is fermented using a starter culture according to claim 1.

8. The method according to claim 7, characterized in that the plant-based milk alternative product is a yogurt alternative product.

9. The method according to claim 7, comprising the following method steps:
provision of a plant-based milk alternative;
adding the starter culture to the plant-based milk alternative;
fermentation of the plant-based milk alternative;
optionally, further processing of the plant-based milk alternative product;
filling and packaging of the plant-based milk alternative product.

10. A fermented plant-based milk alternative product, characterized in that the fermented plant-based milk alternative product is fermented using a starter culture according to claim 1.

11. The fermented plant-based milk alternative product according to claim 10, characterized in that the fermented plant-based milk alternative product is a yogurt alternative product.

12. The fermented plant-based milk alternative product according to claim 10, characterized in that the fermented plant-based milk alternative product has a thickening and/or an acidification and/or a reduced content of off-flavor substances and/or an increased content of milk and/or yogurt flavorings and/or a reduced content of antinutritional ingredients as a result of the fermentation.

13. A method for providing a starter culture with microorganisms for the fermentation of a plant-based milk alternative based on a plant-based protein source from a defined plant-based material, characterized in that the defined plant-based material is used for the isolation of the microorganisms.

14. The method according to claim 13, comprising the following method steps:
provision of a sample of the defined plant material;
enrichment and isolation of microorganisms, in particular gram-positive bacteria, from the sample of plant material;
optionally, genotaxonomic identification of the microorganisms;
optionally, phenotypic characterization of the microorganisms;
performing trial fermentations of the plant-based milk alternative with the isolated microorganisms;
selecting and compiling suitable microorganisms based on the results of the trial fermentations for the provision of a starter culture.

15. The method according to claim 14, characterized in that the phenotypic characterization of the microorganisms comprises at least one of the following aspects:
thickening of a matrix;
acidification of a matrix;
degradation of off-flavor substances, particularly hexanal;
manufacturing of milk and/or yogurt flavorings, in particular acetaldehyde and/or diacetyl and/or acetoin;
usability of different sugar sources;
influence of different sugar sources on metabolite production.

16. A method according to claim 13, characterized in that the defined plant material is plant material from a member of the legume family (Fabaceae), in particular from Pisum sativum and/or Vicia faba.
